(19) United States
(12) Patent Application Publication
(10) Pub. No.: US 2015/0112080 A1
(43) Pub. Date: Apr. 23, 2015

(54) ZINC SALT OF ISOTHIAZOLONE COMPOUND, METHOD FOR REDUCING IRRITATION CAUSED BY ISOTHIAZOLONE COMPOUND, ANTIBACTERIAL AND ANTIFUNGAL METHOD USING ZINC SALT OF ISOTHIAZOLONE COMPOUND, AND ANTIBACTERIAL AND ANTIFUNGAL COMPOSITION

(71) Applicant: NIPPON SODA CO., LTD., Tokyo (JP)

(72) Inventors: Tetsuo KUBOTA, Haibara-gun (JP); Motoaki YABE, Yotsukaido-shi (JP); Shigebumi ARAI, Katori-shi (JP)

(73) Assignee: NIPPON SODA CO., LTD., Tokyo (JP)

(21) Appl. No.: 14/582,397

(22) Filed: Dec. 24, 2014

Related U.S. Application Data
(63) Continuation of application No. 14/141,111, filed on Dec. 26, 2013.
Application No. 14/141,111 is a continuation of application No. 13/067,622, filed on Jun. 15, 2011, now abandoned.
Application No. 13/067,622 is a continuation of application No. 12/452,688, filed on Jan. 27, 2010, now abandoned.

(30) Foreign Application Priority Data
Jul. 19, 2007 (JP) 2007-188105

(51) Int. Cl. C07D 275/04

(52) U.S. Cl. CPC C07D 275/04
USPC 548/108

(57) ABSTRACT

A method for reducing irritation caused by an isothiazolone compound represented by formula (1) by converting the isothiazolone compound represented by formula (1) into a zinc salt.

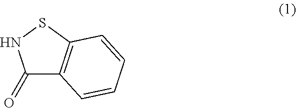

CROSS-REFERENCE TO PRIOR APPLICATION

This is a continuation of application Ser. No. 14/141,111 filed Dec. 26, 2013, which is a continuation of application Ser. No. 13/067,622 filed Jun. 15, 2011, which is a continuation of application Ser. No. 12/452,688 filed Jan. 27, 2010, which is a National Stage Application of PCT/JP2008/062470 filed Jul. 10, 2008, and claims the benefit of Japanese Patent Application No. 2007-188105 filed Jul. 19, 2007. The entire disclosures of the prior applications are hereby incorporated by reference herein in their entirety.

### TECHNICAL FIELD

The present invention relates to a zinc salt of an antibacterial and antifungal isothiazolone compound which has been improved, in particular, regarding skin irritating properties; a method for reducing irritation caused by the isothiazolone compound; an antibacterial and antifungal method using the zinc salt of an isothiazolone compound; and an antibacterial and antifungal composition.

### BACKGROUND ART

Isothiazolone compounds such as 1,2-benzoisothiazolin-3-one (hereafter, referred to as “BIT”) exhibit antibacterial and antifungal activities, and thus have been used in, for example, an emulsion coating material, a synthetic polymeric emulsion, a latex, a metalworking fluid, an adhesive, a thickener, a surfactant solution, pulp white water, and in various other fields.

However, isothiazolone compounds are potent skin and mucosal irritants, and thus they need to be handled with the utmost care and their applications have also been limited.

Therefore, the development of new antibacterial and antifungal agents containing an isothiazolone compound in a form so as to cause minimal skin irritation has been desired.

Regarding the present invention, in Patent Document 1, an antibacterial composition which can be used as an aqueous solution by making BIT into the form of an alkali metal salt has been proposed. As disclosed in this document, by preparing BIT as an alkali metal salt, the problems of potent skin and mucosal irritation caused by the vapor of BIT can be solved. However, the alkali metal salt of BIT or the aqueous solution thereof has not improved the aspect of skin or mucosal irritation at all, and thus the situation where the handling thereof requires the greatest possible care has not been changed.

In Patent Document 2, an antibacterial and antifungal composition formed of an antibacterial and antifungal agent, which is a metal salt of an organic compound where the compound itself has an antifungal activity, and a water-soluble polymeric material, has been disclosed. In this document, as the alkali metal salts of BIT, which itself has an antifungal activity, a silver salt, a copper salt, a zinc salt, or the like has been described.

However, the antibacterial and antifungal compositions described in the working examples of Patent Document 2 were a silver salt of 2-mercaptopyridine-N-oxide, a complex of silver and copper salts of BIT, and a complex of silver, copper and zinc salts of 2-mercaptopyridine-N-oxide, and thus there has been no specific description on a zinc salt of BIT.

In addition, since the silver salt of BIT which is specifically described in Patent Document 2 is readily decomposed by light and thereby forming silver deposition, and also causes strong discoloration of coating materials, it could not be used as a preservative for coating materials. Moreover, the copper salt of BIT had a blue-green color, and when used, for example, as an antibacterial component in a coating material, there was a possibility that the color of a product, onto which the coating material was applied, to deteriorate.

Patent Document 1: Japanese Unexamined Patent Application, First Publication No. Hei 8-3004

Patent Document 2: Japanese Unexamined Patent Application, First Publication No. Hei 11-107162

### DISCLOSURE OF INVENTION

### Problems to be Solved by the Invention

The present invention is made in view of the above circumstances regarding the conventional techniques and its object is to provide a zinc salt of an isothiazolone compound which is free from the problem of coloration and is also stable and causes a reduced level of skin and mucosal irritation while maintaining an excellent level of antibacterial and antifungal activity; a method for reducing the extent of irritation caused by the isothiazolone compound that is a potent skin and mucosal irritant; an antibacterial and antifungal method using the zinc salt of an isothiazolone compound; and an antibacterial and antifungal composition containing the zinc salt of an isothiazolone compound.

### Means for Solving the Problems

The present inventors conducted intensive and extensive studies in order to solve the above-mentioned problems. As a result, they discovered that a zinc salt of BIT, obtained by adding a predetermined amount of the zinc salt to a propylene glycol solution of BIT, is free from the problem of coloration, is stable, and causes a reduced level of skin and mucosal irritation while maintaining an excellent level of antibacterial and antifungal activity, and thus completed the present invention.

As described above, according to a first aspect of the present invention, any one of the following zinc salts (A), (B) and (C) of an isothiazolone compound represented by a formula (1) is provided.

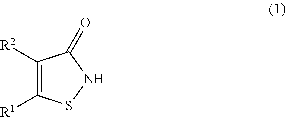

(A) A zinc salt of an isothiazolone compound in which the compound is represented by a formula (1) (In the above formula, R¹ and R² each independently represents a hydrogen atom, a halogen atom, or an alkyl group of 1 to 20 carbon atoms. In addition, R¹ and R² may bind to each other to form a substituted or unsubstituted carbocycle).

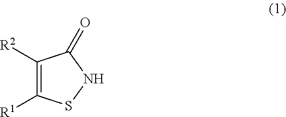

(B) A zinc salt of an isothiazolone compound, in which the compound is represented by the aforementioned formula (1), which is obtained by mixing at least one isothiazolone compound represented by the aforementioned formula (1) (in the formula, R¹ and R² are as defined above) and at least one zinc compound (excluding the zinc salt of an isothiazolone compound, in which the compound is represented by the aforementioned formula (1)) at a predetermined ratio.

(C) The zinc salt according to the above (B) which is obtained by mixing the compound represented by the aforementioned formula (1) and the aforementioned zinc compound so that the ratio between (the compound represented by the aforementioned formula (1)) and (the zinc compound) in terms of a molar ratio is (the compound represented by the aforementioned formula (1)):(the zinc compound)=1:10 to 10:1.

According to a second aspect of the present invention, the following method (D) for reducing the extent of irritation caused by the isothiazolone compound represented by the aforementioned formula (1) is provided.

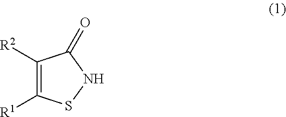

(D) A method for reducing irritation caused by an isothiazolone compound represented by the aforementioned formula (1), the method characterized in that the isothiazolone compound represented by the aforementioned formula (1) is converted into a zinc salt (in the above formula, R¹ and R² each independently represents a hydrogen atom, a halogen atom, or an alkyl group of 1 to 20 carbon atoms. In addition, R¹ and R² may bind to each other to form a substituted or unsubstituted carbocycle).

According to a third aspect of the present invention, the following antibacterial and antifungal method (E) is provided.

(E) An antibacterial and antifungal method characterized by using the zinc salt according to any one of the above (A) to (C).

According to a fourth aspect of the present invention, the following antibacterial and antifungal composition (F) is provided.

(F) An antibacterial and antifungal composition characterized by including at least one of the zinc salts according to any one of the above (A) to (C).

### Effect of the Invention

According to the present invention, a zinc salt of an isothiazolone compound which is free from the problem of coloration and is also stable and causes a reduced level of skin and mucosal irritation while maintaining an excellent level of antibacterial and antifungal activity; a method for reducing the extent of irritation caused by the isothiazolone compound that is a potent skin and mucosal irritant; an antibacterial and antifungal method using the zinc salt of an isothiazolone compound; and an antibacterial and antifungal composition containing the zinc salt of an isothiazolone compound are provided.

### BEST MODE FOR CARRYING OUT THE INVENTION

Hereinafter, the present invention will be described in detail in separate sections: i.e., 1) a zinc salt of an isothiazolone compound represented by formula (1); 2) a method for reducing irritation caused by the isothiazolone compound represented by formula (1); 3) an antibacterial and antifungal method; and 4) an antibacterial and antifungal composition.

1) A Zinc Salt of an Isothiazolone Compound Represented by the Aforementioned Formula (1)

The first aspect of the present invention is a zinc salt of an isothiazolone compound represented by the aforementioned formula (1).

(1) An Isothiazolone Compound Represented by the Aforementioned Formula (1)

In the aforementioned formula (1), R¹ and R² each independently represents a hydrogen atom; a halogen atom such as a fluorine atom, a chlorine atom and a bromine atom; or an alkyl group of 1 to 20 carbon atoms such as a methyl group, an ethyl group, an n-propyl group, an i-propyl group, an n-butyl group, an i-butyl group, an s-butyl group, a t-butyl group, an n-pentyl group, an n-hexyl group, an n-heptyl group, an n-octyl group, an n-nonyl group and n-decyl group.

In addition, R¹ and R² may bind to each other to form a substituted or unsubstituted carbocycle. Examples of such carbocycles include an aromatic ring such as a benzene ring and naphthalene ring; and an aliphatic ring such as a cyclopentane ring, a cyclohexane ring, a cycloheptane ring and a cyclooctane ring.

In addition, examples of the substituent for the aforementioned carbocycles include an alkyl group such as a methyl group and an ethyl group; a halogen atom such as a fluorine atom and a chlorine atom; an alkoxy group such as a methoxy group and an ethoxy group; a nitro group; and a cyano group.

Among various possibilities, in the present invention, as the isothiazolone compound represented by the aforementioned formula (1), 1,2-benzoisothiazolin-3-one is particularly preferable.

(2) A Zinc Salt of an Isothiazolone Compound Represented by the Aforementioned Formula (1)

The present invention is a zinc salt of an isothiazolone compound represented by the aforementioned formula (1) (hereafter, frequently referred to as “isothiazolone compound (1)”).

A zinc salt of an isothiazolone compound (1) according to the present invention can be obtained by reacting the isothiazolone compound (1) with a zinc compound (excluding a zinc salt of the isothiazolone compound (1)) at a predetermined ratio.

There are no particular limitations on the zinc compound used as long as it is a salt of zinc (II). Examples thereof include zinc chloride, zinc bromide, zinc iodide, zinc nitrate, zinc sulfate, zinc acetate and zinc bisacetylacetonate.

The ratio by which the isothiazolone compound (1) and a zinc compound are reacted is such that a molar ratio (isothiazolone compound (1)) and (zinc compound) is (isothiazolone compound (1)):(zinc compound)=1:10 to 10:1, preferably 1:5 to 10:1, and more preferably 1:1 to 10:1. By reacting the isothiazolone compound (1) and a zinc salt at a ratio within such ranges, a target product can be obtained at satisfactory yield.

The reaction between the isothiazolone compound (1) and a zinc compound can be conducted in an adequate solvent. Examples of the solvent used include water; alcohol-based solvents such as methanol, ethanol, propanol, isopropanol and butanol; ketone-based solvents such as acetone, methyl ethyl ketone, methyl isobutyl ketone, diethyl ketone and cyclohexanone; ether-based solvents such as diethyl ether, dipropyl ether, tetrahydrofuran, and 1,2-dimethoxyethane; amide-based solvents such as N,N-dimethylformamide, N,N-dimethylacetamide, N-methylpyrrolidone and hexamethylphosphoric triamide; glycol-based solvents such as glycerin, ethylene glycol, diethylene glycol, dipropylene glycol, polypropylene glycol, propylene glycol, triethylene glycol and hexyl glycol; glycol ether-based solvents such as diethylene glycol, ethylene glycol monoethyl ether, ethylene glycol monobutyl ether, diethylene glycol monomethyl ether and diethylene glycol monoethyl ether; glycol diester-based solvents such as ethylene glycol diacetate; glycol ester ether-based solvents such as ethylene glycol monomethyl ether acetate and diethylene glycol monomethyl ether acetate; ester-based solvents such as methyl acetate, ethyl acetate, 3-methoxybutyl acetate, 2-ethoxymethyl acetate, propylene carbonate and dimethylglutarate. These solvents can be used alone or in combination of two or more types thereof.

Among these, it is particularly preferable to use at least one solvent selected from the amide-based solvents, the glycol-based solvents, the glycol ether-based solvents, the glycol diester-based solvents and the glycol ester ether-based solvents.

There are no particular limitations on the amount of solvent used as long as it is able to completely dissolve an isothiazolone compound, but is preferably within a range from 10 to 100 g with respect to 1 g of the isothiazolone compound (1).

The reaction between the isothiazolone compound (1) and a zinc compound is carried out, for example, by adding the zinc compound or a solvent solution of the zinc compound to a solvent solution of the isothiazolone compound (1) in a predetermined amount, and then stirring the entire mixture.

The reaction temperature is typically within a temperature range from 0° C. to the boiling point of the used solvent, and is preferably within a range from 10 to 80° C.

The reaction time is typically from a few minutes to a few hours, although depending on the scale of the reaction.

After completion of the reaction, the reaction mixture is separated by filtration, and the obtained filtered material is washed with an adequate solvent and then dried, thereby isolating a target product.

A zinc salt of the isothiazolone compound (1) obtained in the above described manner is a compound which is free from the problem of coloration and is stable, and causes a reduced level of skin and mucosal irritation while maintaining an excellent level of antibacterial and antifungal activity.

2) Method for Reducing Irritation

The second aspect of the present invention is a method for reducing the extent of irritation caused by the isothiazolone compound represented by the aforementioned formula (1), the method characterized in that the isothiazolone compound (1) is converted into a zinc salt.

Reduced level of skin and mucosal irritation exhibited by the zinc salt of the isothiazolone compound (1) can be confirmed, for example, by the absence of erythema occurring in the mouse ear auricle or the extent of hyperplasia being less than 0.1 mm, in the mouse local lymph node assay.

3) Antibacterial and Antifungal Method

The third aspect of the present invention is an antibacterial and antifungal method characterized by using the zinc salt of the isothiazolone compound (1) according to the present invention.

In order to carry out the method according to the present invention, at least one kind of the zinc salts of the isothiazolone compound (1) according to the present invention may be used directly as it is, or may also be used, as described later, in the form of an antibacterial and antifungal composition containing at least one kind of the zinc salts of the isothiazolone compound (1) according to the present invention.

4) Antibacterial and Antifungal Composition

The fourth aspect of the present invention is an antibacterial and antifungal composition characterized by including at least one kind of the zinc salt of the isothiazolone compound (1) according to the present invention.

Although there are no limitations on the antibacterial and antifungal composition of the present invention as long as it includes at least one kind of the zinc salts of the isothiazolone compound (1) according to the present invention, it may also be a composition that is mixed or diluted with a solvent or a surfactant.

Examples of the solvent used include amides such as dimethylformamide and diethylformamide; ethers such as ethylene glycol monomethyl ether, ethylene glycol monoethyl ether, ethylene glycol monophenyl ether and diethylene glycol monomethyl ether; alcohols or glycols such as isopropyl alcohol, diethylene glycol, propylene glycol, dipropylene glycol and polypropylene glycol; and carbonates such as propylene carbonate. These solvents can be used alone or in combination of two or more types thereof.

Examples of the surfactants used include nonionic surfactants like fatty acid-based surfactants, such as sucrose fatty acid esters, sorbitan fatty acid esters, polyoxyethylene sorbitan fatty acid esters and fatty acid alkanolamides, higher alcohol-based surfactants, such as polyoxyethylene alkyl ethers and polyoxyethylene alkyl esters, and alkyl phenol-based surfactants, such as polyoxyethylene alkyl phenyl ether; and anionic surfactants like fatty acid-based surfactants, such as sodium fatty acids, potassium fatty acids and sodium α-sulfo fatty acid esters, linear alkylbenzene-based surfactants, such as linear sodium alkylbenzene sulfonate, higher alcohol-based surfactants, such as sodium alkylsulfate esters and sodium alkyl ether sulfate esters, α-olefin-based surfactants, such as sodium α-olefin sulfonate, normal paraffin-based surfactants, such as sodium alkyl sulfonate, and sulfonate-based surfactants, such as lignin sulfonate.

The antibacterial and antifungal composition according to the present invention effectively acts on microorganisms such as Bacillus, Pseudomonas, Micrococcus, Flabobacterium, and Alcaligenes, which easily grow in alkali range.

The used amount of the antibacterial and antifungal composition according to the present invention differs, depending on the types of processes, targets, areas, seasons, or the like where the composition is used, and thus no generalizations can be made. However, in terms of the amount of active ingredient, which is composed of the zinc salt of the isothiazolone compound (1) and is contained in the processes or targets where the composition is used, it may be about 0.00001 to 3% by weight, preferably from 0.000025 to 1.0% by weight, and more preferably from 0.002 to 0.5% by weight.

The antibacterial and antifungal composition of the present invention can be used for various targets, as long as it is used for the bacteriocidal, fungicidal, bacteriostatic or fungistatic purpose. For example, it can be used as an antibacterial and antifungal component for emulsions, coating materials, adhesives, papers, pulps, metalworking fluids, latexes, pigment slurries, rolling oil, slime controllers, thickeners, surfactant solutions, spinning oil, building materials, wood materials, cosmetics, fibers, leathers, or the like.

### EXAMPLES

The present invention will be described in even further detail using Examples below. However, the present invention is in no way limited to the following Examples.

### Example 1

### Synthesis of Zinc Salt of BIT

To a solution prepared by dissolving 34.76 g (0.2 mol) of BIT (product name: Proxel Press Paste (S), manufactured by Arch Chemicals Japan, Inc.) in 500 ml of propylene glycol (PG) heated to 60° C., a solution prepared by dissolving 24.1 g (0.11 mol) of zinc acetate (Wako Pure Chemical Industries, Ltd.) in 200 ml of PG heated to 60° C. was added, and the entire mixture was stirred with cooling for 1 hour.

Then the stirring was stopped, and an opaque substance in the reaction solution was completely precipitated, and the resulting precipitates were then collected by filtration. The precipitates collected by filtration were washed with 500 ml of distilled water, and the resulting washed material was suspended in 200 ml of distilled water.

After confirming that this suspension had a pH of about 6 (confirming that acetic acid was removed by washing), a suction filtration was conducted once more, and the resultant was dried together with a filter paper in a vacuum desiccator, thereby yielding a white solid. It was verified by elemental analysis that the obtained solid was a zinc salt of BIT (hereafter, frequently referred to as BIT/Zn salt).

### Local Lymph Node Proliferation Assay Using a Mouse

### (1) Administration Solution

(A) An administration solution A prepared by suspending the BIT/Zn salt obtained in Example 1 in a mixed solvent, which was composed of N,N-dimethylacetamide, acetone and ethanol and which contained the respective component at a ratio of 4:3:3 (volume ratio) (hereafter, frequently referred to as “DAE”), so as to achieve a 5% by weight solution.

(B) An administration solution B prepared by dissolving BIT in DAE so as to achieve a 5% by weight solution.

### (2) Application of an Administration Solution

12.5 μL of the administration solutions A and B were respectively applied inside and outside the ear auricle of a mouse using a micropipette. This process was carried out for both ears, once a day, and for 3 days. The applied dose was 50 μL/mouse for one day.

### (3) Measurement of Body Weight

Regarding the mouse provided for the experiment, the body weight thereof was measured just before the application on day 0 of the experiment, on day 3 of the experiment, and just before the dissection thereof (on day 6).

### (4) Measurement of Ear Auricle Thickness

Regarding the mouse provided for the experiment, the ear auricle thickness thereof was measured on day 1 and on day 3 of the experiment, using a digital thickness gage manufactured by Teclock Corporation.

The extent of hyperplasia was calculated by the formula: (ear auricle thickness on day 3 of the experiment)—(ear auricle thickness on day 1 of the experiment). When the extent of hyperplasia was 0.1 mm or greater, it was evaluated as “irritating”.

### (5) Observation of Symptoms

During the administration period, the observations were made once just before the administration and once 3 hours after the administration. During other periods, the observations were made once a day. The presence and absence of erythema in the ear auricle was observed just before the administrations on day 1, day 2 and on day 3 of the experiment, and the observations made were evaluated in scores as 0 to 16 grades.

### Method for Evaluating Mouse Ears

Grades 0-9: Evaluated as possibly not irritating to humans.

Grades 10-15: Evaluated as possibly slightly irritating to humans.

Grades greater than grade 15: Evaluated as possibly irritating to humans.

TABLE 1

| | Symptoms | | |
|---|---|---|---|
| | Thickness of ear auricle (mm) | | |
| Tested | Thickness of | Extent of | Grade for |
| substance | ear auricle | hyperplasia | erythema |
| DAE | 0.31 | 0.01 | 0 |
| BIT | 0.39 | 0.10 | 16 |
| BIT/Zn salt | 0.34 | 0.04 | 0 |

As shown in Table 1, the group administered with BIT exhibited the occurrence of erythema which was evaluated as grade 16, thereby confirming that BIT was irritating to the human skin. On the other hand, no erythema was observed in the group administered with DAE which served as a control group and the group administered with the BIT/Zn salt.

Regarding the extent of hyperplasia, it was 0.1 mm in the group administered with BIT, whereas it was 0.04 mm in the group administered with the BIT/Zn salt. Because the group administered with BIT exhibited the extent of hyperplasia of 0.1 mm or greater, which were the criteria for evaluating irritation, it was shown that BIT was irritating.

In addition, in terms of general symptoms, no abnormality was observed in the control group (administered with DAE) or the group administered with BIT. In the case of the group administered with the BIT/Zn salt, attachment of white powder on the ear auricle was observed 3 hours after the administration. However, the white powder was not observed 24 hours after the administration, and erythema was not observed either.

### Thermal Stability Test

A test was carried out on the thermal stability of the BIT/Zn salt obtained in Example 1 and of BIT. As a result, BIT broke down at 300° C. or less, whereas the BIT/Zn salt remained stable even at 400° C.

### Test for Determining Minimum Concentration for Inhibition of Growth

For the test samples, 3 types of samples were used; i.e., the BIT/Zn salt obtained in Example 1 (in the form of a powder containing 79.7% of active ingredients), a BIT material/SC formulation (i.e., Bestcide-NS manufactured by Nippon Soda Co., Ltd. containing 33% of active ingredients), and ZnO (zinc oxide) of 99.0% or greater (manufactured by Wako Pure Chemical Industries, Ltd.). Evaluations were made by conducting an agar plate dilution method using a potato dextrose agar medium. Two fungal strains; i.e., Aspergillus niger NBRC6342 and Cladosporium cladosporioides NBRC6348, were used as microorganisms for the test, and the BIT/Zn salt, BIT and ZnO were used at a concentration of 50 to 1,000 mg/L. The results are shown in Table 2 and Table 3. It should be noted that in Table 3, the results on the analysis of minimum inhibitory concentration (MIC) based on the molecular weights (i.e., MIC values on a molar basis) are shown.

TABLE 2

| | Concentration of active ingredient (mg/L) | | | | | | | | | |
|---|---|---|---|---|---|---|---|---|---|---|
| Sample | 0 | 50 | 100 | 150 | 200 | 250 | 500 | 750 | 1,000 | MIC value |
| BIT/Zn salt | +++ | +++ | +++ | +++ | +++ | +++ | +++ | − | − | 750 |
| ZnO | +++ | +++ | +++ | +++ | +++ | +++ | +++ | +++ | | >1,000 |
| BIT | +++ | +++ | +++ | − | − | − | − | − | − | 200 |

TABLE 3

| | | MIC value (mM/L) | |
|---|---|---|---|
| | | | Cladosporium |
| | Molecular | Aspergillus niger | cladosporioides |
| Sample | weight | NBRC6342 | NBRC6348 |
| BIT/Zn salt | 367.4 | 2.0 | <0.1 |
| ZnO | 81.4 | >12.3 | 6.10 |
| BIT | 151 | 1.3 | <0.3 |

Although MIC values differed depending on the types of microorganisms used, it was confirmed that the activity of the BIT/Zn salt achieved a level which was satisfactory to be put into practice.

### Antibacterial Test

Regarding the BIT/Zn salt obtained in Example 1, an antibacterial test was carried out using a coated film.

For the samples, 2 types of samples were used; i.e., A) the BIT/Zn salt (in the form of a powder containing 79.7% of active ingredients) and B) a BIT material/SC formulation (i.e., Bestcide-NS manufactured by Nippon Soda Co., Ltd. containing 15.0% of active ingredients). For the coating material, an aqueous acrylic emulsion coating material manufactured by Nippe Home Products Co., Ltd. (a preservative-free product) was used.

### (1) Preparation of Test Specimen

To a blank coating material, either A) 0.019% by weight of the BIT/Zn salt or B) 0.1% by weight of the BIT technical product/SC formulation was added, and the resultant was then stirred and mixed. The coating material to which the BIT/Zn salt was added was further subjected to an ultrasonic treatment to prepare a uniform dispersion.

Subsequently, the obtained drug-added coating materials were applied onto both sides (200 g/m³ for each side) of a sterilized filter paper having a size of 50×50 mm, and the resultant was cured at room temperature for 24 hours. Test specimens where no drug was added were also prepared in the same manner.

### (2) Antibacterial Test

The test was carried out in accordance with JIS Z 2801. In other words, two strains of bacteria were used for the test; i.e., Escherichia coli NBRC 3972 (Escherichia coli) and Staphylococcus aureus NBRC 12732 (Staphylococcus aureus), and their cultures preincubated in an NA medium were diluted with a 500-fold diluted NB liquid medium to prepare bacterial suspensions for inoculation. 0.4 ml of the bacterial suspension was inoculated onto each test specimen (i.e., 50×50 mm sized filter paper with a coated film thereon), and the resultant was covered with a covering film (which was already sterilized and having a size of 45×45 mm). The prepared test specimen was put in a petri dish and incubated for 24 hours in a thermo-hygrostat at a temperature of 35±1° C. and a relative humidity of 95%. Thereafter, the sample surface and the covering film were washed using 10 ml of an SCDLP medium, and the number of bacterial cells per 1 ml of the washed out liquid was calculated by counting the number of colonies using an NA medium.

The test results were evaluated based on the following criteria.

1) Control

A coated film obtained using a blank coating material was used as a control.

The number of bacterial cells was observed, just after the inoculation (A), and after 24 hours of incubation (B).

2) Requirements for a Valid Test

1. {(A-B)/A}×100≦90 [A: just after the inoculation, B: after 24 hours of incubation]

2. Results of the viable cell count obtained in (A) were within a range from 1.0 to 5.0×10⁵ cells per petri dish.

3. Results of the viable cell count obtained in the blank (untreated) were 1.0×10³ or more cells per petri dish.

3) Test Results

The test results are shown in the following Table 4. In Table 4, larger values for the antibacterial activity indicate stronger antibacterial activity.

TABLE 4

| | Escherichia coli | | | Staphylococcus aureus | | |
|---|---|---|---|---|---|---|
| | Number of | Logarithmic | Antibacterial | Number of | Logarithmic | Antibacterial |
| Sample name | viable cells | value | activity | viable cells | value | activity |

Substance added experiments

| | | | | | | |
|---|---|---|---|---|---|---|
| BIT/Zn salt | <10 | 1.0 | 4.9 | <10 | 1.0 | 3.6 |
| BIT/SC | <10 | 1.0 | 4.9 | <10 | 1.0 | 3.6 |

Control experiments

| | | | | |
|---|---|---|---|---|
| Just after inoculation (value | 6.7 × 10⁴ | 4.8 | 1.3 × 10⁵ | 5.1 |
| A) | | | | |
| After 24 hours of incubation | 7.3 × 10⁵ | 5.9 | 3.9 × 10⁴ | 4.6 |
| (value B) | | | | |

Unit for viable cell count: Number of cells per petri dish

As shown in Table 4, the BIT/Zn salt exhibited the same level of antibacterial activity as that of BIT, and thus it was verified that formation of a Zn salt does not result in the decline of antibacterial activity.

### INDUSTRIAL APPLICABILITY

By using the zinc salt of the isothiazolone compound according to the present invention, an antibacterial and antifungal composition which is free from the problem of coloration and is stable, and causes a reduced level of skin and mucosal irritation while maintaining an excellent level of antibacterial and antifungal activity can be provided.

1. A method for reducing irritation caused by an isothiazolone compound represented by formula (1): 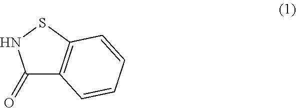
the method comprising: converting the isothiazolone compound represented by formula (1) into a zinc salt.

2. The method according to claim 1, further comprising using at least one solvent selected from the group consisting of water, alcohol-based solvents, ketone-based solvents, ether-based solvents, amide-based solvents, glycol-based solvents, glycol ether-based solvents, glycol diester-based solvents, glycol ester ether-based solvents, and ester-based solvents.

3. The method according to claim 1, further comprising mixing at least one isothiazolone compound represented by formula (1) and at least one zinc compound, excluding the zinc salt of an isothiazolone compound represented by formula (1), at a predetermined ratio.

4. The method according to claim 2, further comprising mixing at least one isothiazolone compound represented by formula (1) and at least one zinc compound, excluding the zinc salt of an isothiazolone compound represented by formula (1), at a predetermined ratio.

5. The method according to claim 3, wherein the zinc salt is obtained by mixing the compound represented by formula (1) and the zinc compound so that a molar ratio between the compound represented by the formula (1) and the zinc compound is from 1:10 to 10:1.

6. The method according to claim 4, wherein the zinc salt is obtained by mixing the compound represented by formula (1) and the zinc compound so that a molar ratio between the compound represented by the formula (1) and the zinc compound is from 1:10 to 10:1.
